(19) United States
(12) Patent Application Publication
(10) Pub. No.: US 2020/0305789 A1
(43) Pub. Date: Oct. 1, 2020

(54) SYSTEM AND METHOD FOR ASSESSING ADVANCED KINETIC SYMPTOMS

(71) Applicant: Global Kinetics Pty Ltd, Melbourne (AU)

(72) Inventor: Malcolm Kenneth Horne, Melbourne (AU)

(73) Assignee: Global Kinetics Pty Ltd, Melbourne (AU)

(21) Appl. No.: 16/307,669

(22) PCT Filed: Jun. 6, 2017
(86) PCT No.: PCT/AU2017/050555

§ 371 Date: Dec. 6, 2018

(30) Foreign Application Priority Data
Jun. 6, 2016 (AU) 2016902203

(51) Int. Cl. A61B 5/00; A61B 5/11; G16H 10/60; G16H 15/00; G16H 40/67; G16H 80/00

(52) U.S. Cl. CPC A61B 5/4842; A61B 5/4082; A61B 5/1101; A61B 5/4839; A61B 5/6802; A61B 2562/0219; A61B 5/7465; G16H 10/60; G16H 15/00; G16H 40/67; G16H 80/00; A61B 5/0022

(57) ABSTRACT

A method of determining a state of progression in a subject of a disease or treatment having motion symptom comprises obtaining a time series of motion data from a motion detector worn on an extremity of the subject, over an extended period during usual activities of the subject; processing the motion data to produce a plurality of measures of kinetic state of the subject at a respective plurality of times throughout the extended period, each measure of kinetic state comprising at least one of: a measure for bradykinesia, and a measure for dyskinesia; determining a measure of dispersion of the measures of kinetic state; combining the measure of dispersion with at least one other data characteristic determined from the motion data, to produce a selection score; and generating an output indicating the selection score.

The present application claims priority from Australian Provisional Patent Application No. 2016902203 filed on 6 Jun. 2016, the content of which is incorporated herein by reference.

### TECHNICAL FIELD

The present invention relates to determining and/or monitoring a state of progression in a subject of a disease or treatment having motion symptoms, by analysing the kinetic state of the subject, and in particular the invention relates to a method and system for monitoring bradykinesia and/or dyskinesia to assess the state of progression of the disease or treatment.

### BACKGROUND OF THE INVENTION

A range of diseases, medications, trauma and other factors can lead to a person having motion symptoms. Motion symptoms include dyskinesia, in which the person is in a hyperkinetic state, and bradykinesia, in which the person is in a hypokinetic state.

Bradykinesia is a symptom of dysfunction of the basal ganglia, and any conditions that can affect this part of the brain will cause bradykinesia. Similarly any condition which has a hyperdopaminergic state or excess activity of the basal ganglia will produce hyperkinetic syndromes that include dyskinesia. Hyperkinetic activity can also be seen in other conditions of basal ganglia overactivity such as Tourettes syndrome and Huntingtons disease. For example, bradykinesia is a key manifestation of Parkinson's disease (PD). Dopa, or Levodopa, is often administered to patients having Parkinson's disease, and can have the effect of causing the patient to become dyskinetic for a period of time after administration. However, even for new patients the half-life of levodopa is only of the order of about 90 minutes, so that observed symptoms fluctuate markedly throughout the course of a day. As Parkinson's disease progresses, the half life of L-Dopa shortens and the effective dose range decreases, exacerbating the fluctuations and making dosage control extremely difficult and complex. This is commonly managed by increasing the dose frequency, sometimes to as many as ten doses each day in an attempt to control symptoms and enable the patient to have a reasonable quality of life. Thus, patients with Parkinson's disease may experience periods of bradykinesia, dyskinesia and normal motor function several times a day and throughout the course of a single dose of L-Dopa.

Even if a satisfactory dosage regime is reached at one point in time, the progressive nature of Parkinson's disease means that neurologists must regularly review a patient's symptoms in order to effectively control the patient's ongoing treatment dosage. Without objective and ongoing monitoring it is very difficult for physicians to avoid prescribing either an excessive dose which overly increases episodes of dyskinesia, or an inadequate dose which does not reduce or prevent episodes of bradykinesia. Furthermore conventional clinical treatment relies on a subjective assessment undertaken by a physician as well as historical input from the patient or a witness such as a carer, and gives no objective measure as to the severity of the state or as an indication of whether a change in dose was effective in improving symptoms.

Further, clinical observation typically only occurs over a short period of patient attendance, usually of the order of tens of minutes, once every 3 or 6 months. Fluctuations in kinetic state can be considerable throughout the day and from one day to the next, which significantly complicates attempts at assessing the patient's kinetic state. Clinicians often rely on the patient's recollection and/or written diaries to gain an understanding of the ongoing kinetic state of the patient between clinical appointments. However patients can rarely give objective data, and the effect of a kinetic episode itself can often make it difficult for a patient to make any record whatsoever of the nature of and timing of motor fluctuations.

As Parkinson's disease progresses, and the adequacy of Levodopa treatment to minimise fluctuations attenuates, clinicians look to advanced therapies for treating symptoms of Parkinson's disease. Advanced therapies are required when fluctuations in bradykinesia and dyskinesia caused by the shortened duration of effect and variable absorption of an oral therapy cannot be adequately controlled by such therapy. Such advanced therapies include deep brain stimulation (DBS), apomorphine by continuous infusion, and levodopa-carbidopa (duodopa) intestinal gel. However, in practice recognition of suitable candidates requires considerable expertise and time to accurately identify whether advanced therapy should be initiated for a particular patient. This expertise is based on experience.

In the case of DBS for example, it has been shown that DBS is an effective treatment of PD, for well-selected PD patients. In particular, the period of time in the course of a patient's disease when DBS is indicated is relatively constrained: there is a finite window between the onset of fluctuations and the time when DBS is contraindicated and can cause harm. Thus, accurate patient selection is crucial to ensure positive outcomes. Patient selection for DBS usually happens in two steps. First, the general neurologist who provides routine care for a patient, identifies them as a potential DBS candidate and refers him/her to a Movement Disorder Centre experienced in DBS. Second, at the Movement Disorder Centre, a team of specialists led by a movement disorders neurologist with expertise in selection and management of DBS patients, determines whether a DBS therapy is recommended for the patient. The Movement Disorder Specialist providing the second step is regarded as the “gold standard” for identifying DBS candidates. However, general neurologists who are not expert in movement disorders have difficulty in deciding when to refer patients for consideration for DBS surgery and frequently overlook suitable candidates. Additionally, Movement Disorder Specialists also frequently “miss” candidates as candidates are unable to make the specialists aware of their symptoms. This deprives patients who are suitable for DBS of the opportunity of being assessed in the second stage and gaining a benefit from DBS therapy. Although less common cases that are referred, are referred too late in the window, described above, or as the window is closing. This imposes an unnecessary burden on both the DBS surgical centres, and the patients and their caregivers who undergo unnecessary visits and tests.

Moreover, the second stage of DBS patient selection noted above typically involves a comprehensive selection process requiring considerable resources, the process including levodopa challenge assessment, brain Magnetic Resonance Imaging (MRI) and evaluation of neuropsychological and psychiatric functions.

Any discussion of documents, acts, materials, devices, articles or the like which has been included in the present specification is solely for the purpose of providing a context for the present invention. It is not to be taken as an admission or a suggestion that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect the present invention provides a method of determining a state of progression in a subject of a disease or treatment having motion symptoms, the method comprising:

obtaining a time series of motion data from a motion detector worn on an extremity of the subject, over an extended period during usual activities of the subject;

processing the motion data to produce a plurality of measures of kinetic state of the subject at a respective plurality of times throughout the extended period, each measure of kinetic state comprising at least one of: a measure for bradykinesia, and a measure for dyskinesia;

determining a measure of dispersion of the measures of kinetic state;

combining the measure of dispersion with at least one other data characteristic determined from the motion data, to produce a selection score; and

generating an output indicating the selection score.

According to a second aspect, the present invention provides a non-transitory computer readable medium for determining a state of progression in a subject of a disease or treatment having motion symptoms, comprising instructions which, when executed by one or more processors, causes performance of the following:

obtaining a time series of motion data from a motion detector worn on an extremity of the subject, over an extended period during usual activities of the subject;

processing the motion data to produce a plurality of measures of kinetic state of the subject at a respective plurality of times throughout the extended period, each measure of kinetic state comprising at least one of: a measure for bradykinesia, and a measure for dyskinesia;

determining a measure of dispersion of the measures of kinetic state;

combining the measure of dispersion with at least one other data characteristic determined from the motion data, to produce a selection score; and

generating an output indicating that the selection score.

According to a third aspect the present invention provides a system for determining a state of progression in an subject of a disease or treatment having motion symptoms, the system comprising:

a motion detector configured to be worn on an extremity of the subject and to output a time series of motion data over an extended period; and

a processor configured to receive the motion data and to process the motion data to produce a plurality of measures of kinetic state of the subject at a respective plurality of times throughout the extended period, each measure of kinetic state comprising at least one of: a measure for bradykinesia, and a measure for dyskinesia; the processor further configured to determine a measure of dispersion of the measures of kinetic state; the processor further configured to combine the measure of dispersion with at least one other data characteristic determined from the motion data, to produce a selection score; and the processor further configured to generate an output indicating the selection score.

The invention is computer implemented such that processing steps are performed by a processor such as may be incorporated into a remote server or central computing facility, client computer, mobile device or other processing means configurable for receiving the time series of motion data and producing the plurality of measures of kinetic state of the subject, determining the measure of dispersion and combining the measure of dispersion with at least one other data characteristic which may be determined by the processor from the motion data or from other data. supplied to the processor, to produce a selection score. The processor may also perform a comparison between the selection score and the threshold to generate an output which is indicative of the stage of the motion symptoms.

Thus, in some aspects the present invention provides a computer implemented method for automated determination of a state of progression in an subject of a disease or treatment having motion symptoms, the method comprising; obtaining at a processor a time series of motion data from a motion detector worn on an extremity of the subject, over an extended period during usual activities of the subject; the processor processing the motion data to produce a plurality of measures of kinetic state of the subject at a respective plurality of times throughout the extended period, each measure of kinetic state comprising at least one of: a measure for bradykinesia, and a measure for dyskinesia; the processor determining a measure of dispersion of the measures of kinetic state; and the processor combining the measure of dispersion with at least one other data characteristic determined from the motion data, to produce a selection score; and generating an output indicating the selection score.

In some embodiments of the aspects of the invention, an output is generated indicating that motion symptoms are at an initial stage if the selection score is less than a threshold, and generating an output indicating that motion symptoms are at an advanced stage if the selection score is greater than the threshold.

The measure of dispersion could in some embodiments be a measure of the numerical distance between a high and low percentile of the measures of kinetic state, and for example may be a measure of the interquartile range of the measures of kinetic state. Alternatively, the measure of dispersion could be a measure of the variance of the measures of kinetic state. Alternatively, the measure of dispersion could be a measure of the standard deviation of the measures of kinetic state, or other indicator of the variability, scatter, or spread of the measures of kinetic state.

At least one data characteristic may, in some embodiments comprise a probabilistic measure of bradykinesia. The probabilistic measure of bradykinesia could for example comprise one or more of a mean or median value of a time series of individual measures of bradykinesia obtained throughout an observation period, referred to as BK₅₀, and a 75^{th} percentile value of a time series of individual measures of bradykinesia, referred to as BK₇₅, or any other suitable percentile value BK, of the BKS scores.

Additionally or alternatively, at least one other data characteristic may in some embodiments comprise a probabilistic measure of dyskinesia. The probabilistic measure of dyskinesia could for example comprise one or more of a mean or median value of a time series of individual measures of dyskinesia obtained throughout an observation period, referred to as DK₅₀, and a 75^{th} percentile value of a time series of individual measures of dyskinesia, referred to as DK₇₅, or any other suitable percentile value DKₙ of the DK scores.

Additionally or alternatively, at least one other data characteristic may in some embodiments comprise a median or mean DK score specifically in the period where the subject is ‘off’, that is the period when BK is high. In some embodiments, the at least one other data characteristic may comprise mean BK.

Additionally or alternatively, the at least one other data characteristic may in some embodiments comprise a dosage measure, such as a number of medication reminders prescribed for that subject during a period of interest, such as reminders per day. Such embodiments reflect that increasing number of doses per day or per period correlate with advanced disease state in PD in particular. For example the dosage measure may comprise a binary measure which is 0 for 5 or less doses and 1 for more than 5 daily doses. Or the dosage measure may be zero for subjects prescribed 5 or less doses, and set to [doses—5] for subjects prescribed more than 5 daily doses. The system may in some embodiments deduce the number of prescribed doses from a number of reminders programmed to be delivered by a body-worn device bearing the motion detector.

Additionally or alternatively, the at least one other data characteristic may in some embodiments comprise the proportion of time immobile (PTI) or amount of time immobile (ATI). The PTI/ATI may be deduced from periods when a bradykinesia score (BKS) is very high, such as when BKS exceeds a threshold in the range of 50-100, for example a threshold of 80, during the observation period. The PTUATI can be considered a proxy for day time sleep and cognition

Additionally or alternatively, the at least one other data characteristic may in some embodiments comprise a measure of tremor derived from the motion data.

Additionally or alternatively, the at least one other data characteristic may in some embodiments comprise BKS_{IQR} being the interquartile range of BK scores. Such embodiments recognise that even though the measure of dispersion may be derived wholly or partly from the dispersion of DK scores, the BKS_{IQR} remains an important further indicator of advanced disease state in PD.

Additionally or alternatively, at least one other data characteristic may in some embodiments comprise minutes in bradykinesia, being the number of minutes during an observation period (for example comprising the hours between 09:00 and 18:00) when the subject was bradykinetic. The number of minutes in bradykinesia may be deduced e.g. from the number of minutes when the BKS is above a threshold, or from the number of minutes above 75th percentile of the BKS calculated using the binomial theorem. For example, this measure may seek windows having a small probability of occurrence and define such windows as contributing to a Minutes_Under measure, which is indicative of the minutes in bradykinesia. The window may comprise 7 consecutive BK scores, and assess whether 5 or more of those 7 scores exceed the 75^{th} percentile of all BK scores, noting that the binomial theorem indicates less than a 5% chance of such an occurrence. Such windows may thus be consistent with under-medicadtion of PD.

Additionally or alternatively, the at least one other data characteristic may in some embodiments comprise a measure referred to as Under_Count, comprising a measure of the number of windows of time throughout the observation period in which at least 5 out of 7 BK scores exceed the 75^{th} percentile, or a comparable low percentage event occurs in the BK scores.

Additionally or alternatively, at least one other data characteristic may in some embodiments comprise minutes in dyskinesia, being the number of minutes during an observation period (for example comprising the hours between 09:00 and 18:00) when the subject was dyskinetic. The number of minutes in dyskinesia may be deduced e.g., from the number of minutes when the DKS is above a threshold, or from the number of minutes above the 75th percentile of the DKIS calculated using the binomial theorem. For example this measure may seek windows having a small probability of occurrence and define such windows as contributing to a Minutes_Over measure which is indicative of the minutes in dyskinesia. This window may comprise 7 consecutive DK scores, and assess whether 5 or more of those 7 scores exceed the 75^{th} percentile of all DK scores, again noting that the binomial theorem indicates less than a 5% chance of such an occurrence. Such windows may thus be consistent with over-medication of PD.

Additionally or alternatively, the at least one other data characteristic may in some embodiments comprise a measure referred to as Over_Count, comprising a measure of the number of windows of time throughout the observation period in which at least 5 out of 7 DK scores exceed the 75^{th} percentile, or a comparable low percentage event occurs in the DK scores.

Additionally or alternatively, the at least one other data characteristic may in some embodiments comprise a means of assessing the number of minutes during an observation period when the subject was not dyskinetic or when dyskinesia was below a threshold AND when the subject was not bradykinetic or when bradykinesia was below a threshold. In some embodiments, at least one other data characteristic may comprise data not derived from the motion data and indicative of other factors such as years with motion disease (such as PD), subject's cognitive state, age blood pressure, impulsivity, apathy and the like. These may be collected using a data input device configured to be operable with the inventive system, such as a mobile phone or tablet operated by a nurse or the patient, or it may be supplied to the inventive system along with PKG data by the patient's clinic. The observation period could for example comprise the hours between 09:00 and 18:00.

The extended period may comprise one day, or more than one day, and for example may comprise 6, 7, 8, 9 or 10 days or more. During the extended period, the motion data is preferably obtained for the present method only during waking hours, for example during the period between 9:00 AM and 6:00 PM for the or each day during the extended period, or during an adaptive awake period defined by the absence of sleep using an automatic measure of somnolence.

The present invention recognises that, in Parkinson's disease for example, an increase in a subject's fluctuation between ON, OFF and dyskinetic states (EDS), when further combined with probabilistic bradykinesia and/or dyskinesia measures, is an improved predictor of the subject needing advanced therapies. Thus, monitoring the selection score and in some specific cases, monitoring for elevation of the selection score so produced beyond a threshold provides for an automated and objective method for monitoring progression of the disease and medication/therapy response and/or screening candidates for suitability for advanced therapies.

In some embodiments, each measure of kinetic state comprises both a measure for bradykinesia and a measure for dyskinesia. In such embodiments, the measure of dispersion may be produced as a weighted sum of a measure of the dispersion of the measures for bradykinesia and a measure of the dispersion of the measures for dyskinesia. The weights may each be 0.5, or any other weight in the range of −1 to 1, inclusive although it is contemplated that any weighting scale may be utilised. in alternative embodiments, the measure of dispersion may be produced by summing each measure of bradykinesia with a contemporaneous measure of dyskinesia to produce a combined measure of kinetic state, and determining the measure of dispersion from the dispersion of the combined measures of kinetic state.

In some embodiments, the measure of dispersion may be produced by first processing the measure of the dispersion of the measures for bradykinesia and/or the measure of the dispersion of the measures for dyskinesia using a mathematical function or respective mathematical functions. The processed measures in some embodiments may then be summed linearly to produce the measure of dispersion. The or each mathematical function may comprise applying a weighting as discussed above and/or may comprise applying a logarithmic or exponential to the respective measures, for example.

Combining the measure of dispersion with the at least one other data characteristic may comprise linearly summing, or weighted summing, to produce the selection score. Additionally or alternatively the measure of dispersion and/or the at least one other data characteristic may be modified by any suitable mathematical function prior to or during the combining step, such as by applying a logarithmic or exponential to the measure of dispersion and/or the at least one other data characteristic. The measure of dispersion combined with the at least one other data characteristic may alternatively/additionally produce the selection score visually as a chart or vector.

In some embodiments the method of the present invention may simply determine a selection score. In other embodiments, the invention may determine whether the selection score exceeds the threshold, to give a binary output. However, alternative embodiments may further comprise recording the value of the selection score as determined on different occasions in order to monitor progression of the selection score, for example over the course of hours, days, weeks, months or years. Some embodiments may additionally or alternatively monitor a rate of change in the selection score over time, for example to project or predict disease progression towards a threshold at which advanced therapies may become appropriate. Moreover, monitoring the selection score during progression of a disease may in some embodiments be used as a basis to indicate which therapy, of a plurality of available progressions in therapy, is suitable for that particular patient. Where the selection score is determined repeatedly throughout the course of a single dose of medication, the selection score may take values both above and below a threshold for referral for advanced therapy. Some embodiments may preferentially, or solely, consider selection score values obtained when medication is wearing off or worn off, as a basis for assessing whether advanced therapy is indicated.

The threshold against which the selection score may be compared in order to determine whether the subject might require advanced therapy may be determined or predefined in any suitable manner. For example, the threshold may be predefined as being the median value of the selection score for normal subjects (being subjects not having a neurodegenerative disorder), or the median level of the selection score for subjects having received advanced therapy, or the 75th percentile level of the selection score for subjects having received advanced therapy, or a scalar, logarithmic or exponential variant derived from such values, or the like. In some embodiments, the threshold may be defined by reference to a score or range of scores corresponding to baseline data obtained from the subject being assessed prior to development of a neurodegenerative disorder. In some embodiments, a change in score, or rate of change of scores obtained over time may be utilised as at least one other characteristic used to determine the selection score.

In some embodiments the selection score may be derived from data characteristic of dyskinesia alone, given the importance of dyskinesia to assessing late stage PD or advanced therapy eligibility.

The dyskinesia data may in some embodiments be scores produced in accordance with the teachings of International Patent Application Publication No. WO 2009/149520, the content of which is incorporated herein by reference. The bradykinesia scores may in some embodiments be produced in accordance with the teachings of WO 2009/149520.

An output, indicating that motion symptoms are either at an initial stage or at an advanced stage, may in preferred embodiments be communicated to a physician in order that the physician may consider whether the patient is ready (or not yet ready) to prescribe or administer an altered or advanced therapy based on an objective evaluation of motor criteria. For example, the output may be provided to a general neurologist undertaking a first stage of DBS patient selection, and/or to a DBS specialist undertaking a second stage of DBS patient selection. Such embodiments may thus utilise the selection score to provide a quantitative, simple, automatic, and accurate patient screening tool to support general neurologists in the referral stage, and/or DBS specialists in the DBS eligibility assessment of the subject,

The selection score may additionally or alternatively be used to guide refinements to dosages, whether dosages of medicaments, or dosages or characteristics of DBS stimuli for a DBS recipient. An advanced stage of motion symptoms identified by the present invention may thus indicate a need for revised dosage, mix or selection of oral medicaments, such as an advance from levodopa to a levodopa-carbidopa mix, and/or may indicate a need for advanced therapies such as deep brain stimulation (DBS), apomorphine by continuous infusion, drug delivery by a patch, and levodopa-carbidopa intestinal gel delivered by pump, and/or may indicate any other suitable progression or change in therapy.

According to a further aspect the present invention provides a method of screening an subject having motion symptoms for an advanced therapy, the method comprising:

obtaining a time series of motion data from a motion detector worn on an extremity of the subject, over an extended period during usual activities of the subject;

processing the motion data to produce a plurality of measures of kinetic state of the subject at a respective plurality of times throughout the extended period, each measure of kinetic state comprising at least one of: a measure for bradykinesia, and a measure for dyskinesia;

determining a measure of dispersion of the measures of kinetic state; and

combining the measure of dispersion with at least one other data characteristic determined from the motion data, to produce a selection score; and

generating an output indicating the selection score.

In some embodiments, the method includes generating an output indicating that motion symptoms are at an initial stage if the selection score is less than a threshold, and generating an output indicating readiness for an advanced therapy if the selection score is greater than the threshold. Particularly preferred embodiments for screening a subject having motion symptoms are directed to determining the subject's suitability for an advanced therapy selected from the group including apomorphine therapy, duodopa therapy, and deep brain stimulation (DBS).

According to a further aspect the present invention provides a method for automated screening of a subject to determine clinical readiness to receive advanced therapy for a disease having motion symptoms, the method comprising:

obtaining at a processor a time series of motion data from a motion detector worn on an extremity of the subject, over an extended period during usual activities of the subject;

the processor calculating from the motion data a plurality of measures of kinetic state of the subject at a respective plurality of times throughout the extended period, each measure of kinetic state comprising at least one of: a measure for bradykinesia, and a measure for dyskinesia;

the processor determining a measure of dispersion of the measures of kinetic state; and

the processor combining the measure of dispersion with at least one other data characteristic determined from the motion data, to produce a selection score; and

the processor generating an output indicating one or more of clinical readiness for advanced therapy when the selection score is greater than a threshold; and clinical unreadiness for advanced therapy when the selection score is less than the threshold.

In some embodiments, the threshold is selected from the group including: (i) a median level of the selection score for subjects having received advanced therapy; (ii) the 75th percentile level of the selection score for subjects having received advanced therapy; and (iii) a scalar, logarithmic or exponential variant derived from such values in (i) or (ii).

In some embodiments, the method further includes the step of automatically determining a subject's readiness to receive an advanced therapy selected from the group including: deep brain stimulation (DBS), apomorphine and levodopa-carbidopa (duodopa). Typically, the subject's readiness to receive the selected advanced therapy is automatically determined by the processor when the selection score is greater than a threshold determined by a median level of the selection score for subjects having received the selected advanced therapy, or the 75th percentile level of the selection score for subjects having received the selected advanced therapy or an aggregate of these, or a scalar, logarithmic or exponential variant derived from such values.

In some embodiments, the invention further provides for automatically generating a patient specific report based on a report module containing instructions that are executable by a processor receiving at least the motion data, wherein the report module populates fields of a report template with the selection score and clinical observations derived from the motion data. The report template may include fields selected from the group including: a subject identifier; referring clinician; duration of data collection; dates of data collection; dosage acknowledgements by the subject; therapies prescribed to the subject; dosage reminders provided to the subject; summary of kinetic behaviour during data collection (including one or more of bradykinetic, dyskinetic and tremor motion); summary of kinetic behaviour response to medication; and summary of clinical findings based on at least one of the motion data and measures of dispersion and selection score calculated by the processor.

In some embodiments of the various aspects of the present invention, the invention may further comprise aggregating selection scores obtained for a plurality of subjects in order to assess a state or progression of disease or treatment of the group. Such embodiments may for example be utilised to assess a geographical region or jurisdiction, a clinic, a clinician, or a class of patients, for example to assess whether a country, a region, a clinic or an individual clinician is over- or under-treating their patients, or is prescribing advanced therapies at an appropriate time as compared to other countries, regions, clinics or clinicians.

The motion detector may comprise an accelerometer, outputting acceleration data, or a gyroscope or the like.

It is to be understood each of the various aspects described herein may incorporate features, modifications and alternatives described in the context of one or more other aspects, such as but not limited to the various kinetic states, measures of dispersion and other data characteristics used in the determination of a selection score, :For efficiency, such features, modifications and alternatives have not been repetitiously disclosed for each and every aspect although one of skill in the art will appreciate that such combinations of features, modifications and alternatives disclosed for some aspects apply similarly for other aspects and are within the scope of and form part of the subject matter of this disclosure.

### BRIEF DESCRIPTION OF THE DRAWINGS

An example of the invention will now be described with reference to the accompanying drawings, in which:

FIG. 1 is a diagrammatic view of a means for detection of various Parkinsonian clinical states in accordance with an embodiment of the invention;

FIGS. 2a-2c illustrate a system for kinetic state monitoring and reporting in accordance with one embodiment of the invention. FIG. 2d is an example of a report template that in some embodiments, may be populated automatically with meaningful clinical information and summaries;

FIG. 3 gives an example of DK and BK scores output from one day of wrist-worn data logger motion recording from a patient;

FIGS. 4-6 illustrate the classification efficacy of a selection score in accordance with one embodiment of the invention;

FIG. 7 illustrates the classification efficacy of a selection score in accordance with another embodiment of the invention;

FIG. 8 illustrates the classification efficacy of a selection score in accordance with a further embodiment of the invention;

FIG. 9 illustrates steps in a method of determining a state of progression in a subject of a disease or treatment having motion symptoms according to embodiments of the invention; and

FIG. 10 illustrates a general-purpose computing device that may be used in an exemplary system for implementing the invention.

### DESCRIPTION OF THE PREFERRED EMBODIMENTS

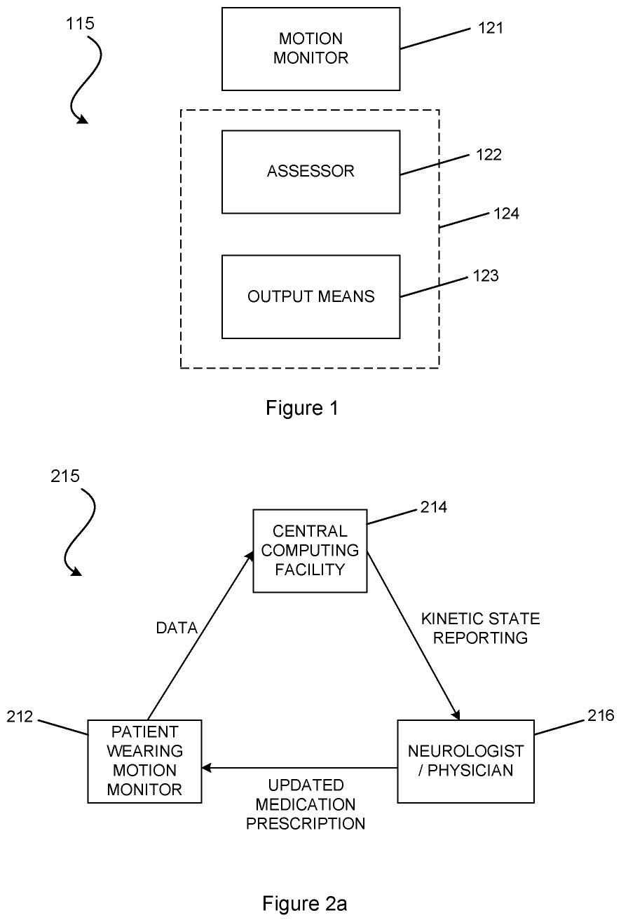

FIG. 1 is a diagrammatic view of apparatus for detection of various Parkinsonian or kinetic states in accordance with an embodiment of the invention. The apparatus 115 comprises three elements for obtaining movement data of a limb of a person to determine a state of progression of the disease or treatment. The apparatus 115 comprises a motion monitor 121 in the form of an accelerometer, an assessor 122 for analysis of the motion monitor data in a manner that provides an objective determination of bradykinesi a and dyskinesia, and an output means 123 for outputting objective determination of bradykinesia or dyskinesia over time periods so as to allow a clinician to prescribe medications or to allow the person to better understand their own kinetic state. Typically, the objective determination is in the form of a selection score.

The motion monitor 121 is a light weight device which is intended to be worn on the most affected wrist of the person to provide a sufficiently accurate representation of the kinetic state of the whole body throughout waking hours. The device is mounted on an elastic wrist band so as to be firmly supported enough that it does not wobble on the arm and therefore does not exaggerate accelerations. The device is configured to rise away from the person's wrist by a minimal amount so as to minimise exaggeration of movements. The motion monitor may be disposable.

The motion monitor 121 records acceleration in three axes X, Y, Z over the bandwidth 0-10 Hz, and stores the three channels of data in memory on-board the device. This motion monitor 121 typically has 1 GB or more of storage so as to allow data collected by the device over an extended period of 6, 7. 8, 9 or 10 or more days to be stored, after which the data can be downloaded and analysed. Data may be downloaded from the motion monitor 121 via a physical connection such as a USB connector or other standard or bespoke means, or by a wireless interface as would be understood by one of skill in the art.

Data may be collected by the motion monitor 121 for a finite assessment period of e.g. 6, 7, 8, 9 or 10 or more days, or it may be collected for analysis by assessor 122 on an ongoing basis, indefinitely, with the motion data being periodically downloaded for evaluation. Periodical removal of the motion monitor 121 from the wearer for download of data also provides an opportunity for the motion monitor 121 to be recharged.

The output means 123 is typically remote from the motion monitor 121 and may be provided in concert with the assessor 122 (as shown by broken line 124) to provide evaluation and selection scores and reports to clinicians. It is to be understood, however, that in some embodiments the motion monitor 121, assessor 122 and optionally the output means 123 may be provided in a single body-worn device.

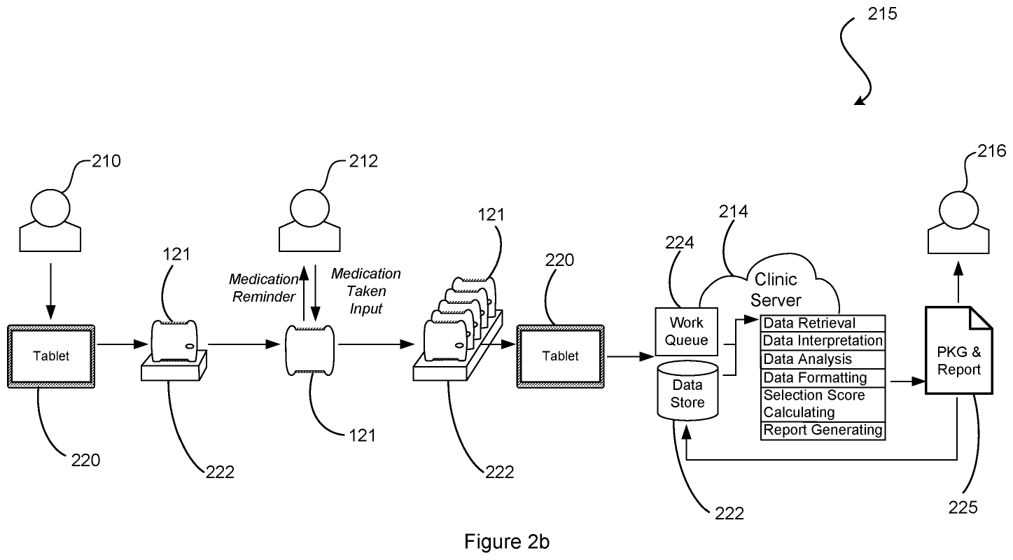

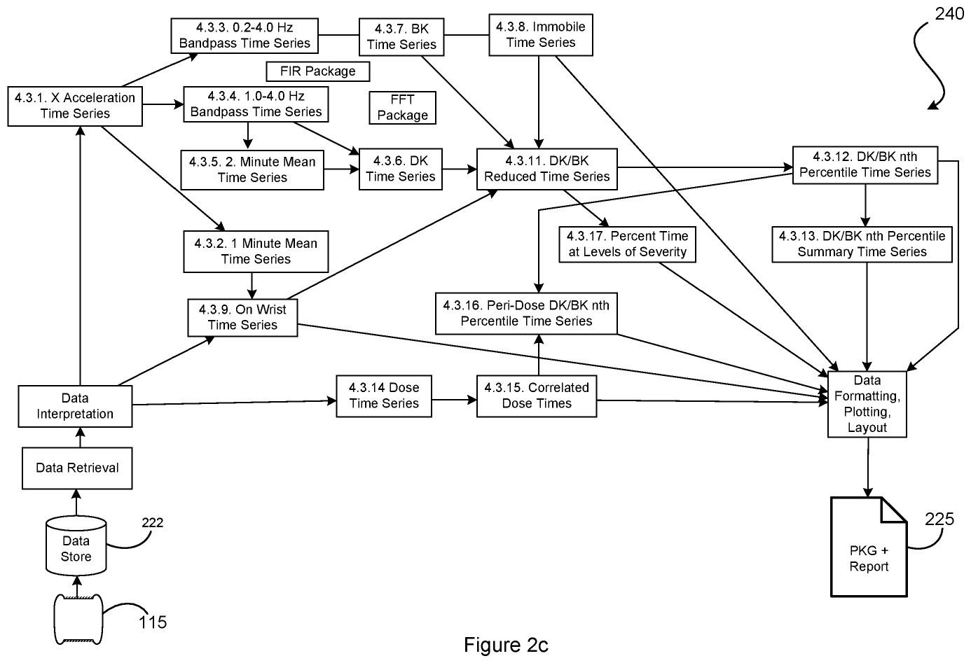

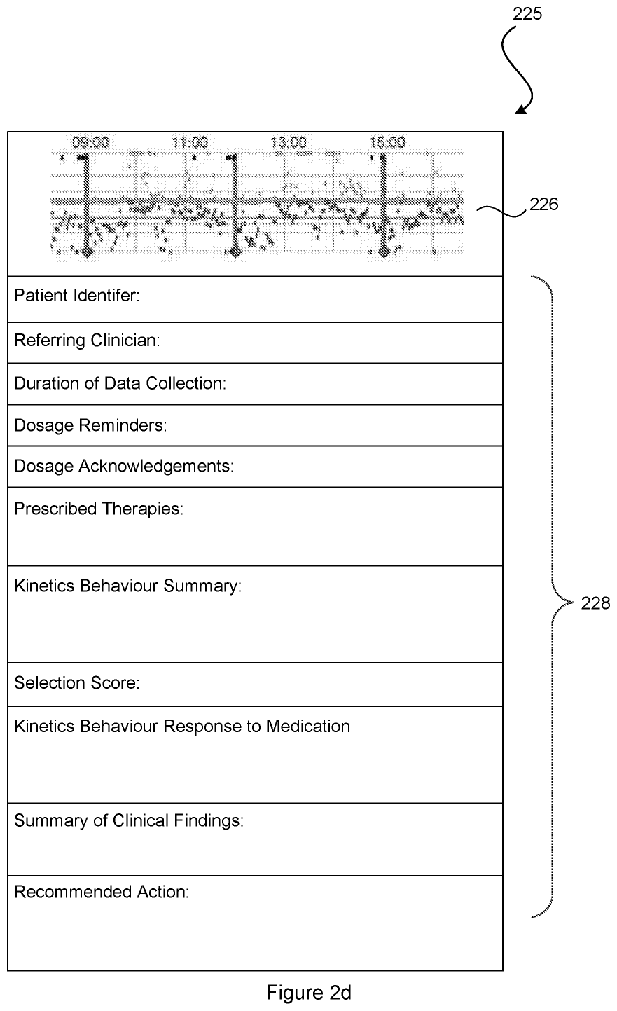

FIGS. 2a-2c illustrate a system 215 for kinetic state monitoring and reporting in accordance with one embodiment of the invention. Referring first to FIG. 2a, a patient 212 is wearing the motion monitor 121 of FIG. 1 which is typically in the form of an accelerometer data logger. The motion monitor 121 logs accelerometer data and communicates it to a central computing facility 214. The computing facility 214 analyses the data to produce an output comprising kinetic state reporting, indicating whether motion symptoms are at an initial stage or an advanced stage. The output is reported to a neurologist or physician 216 who is typically located remotely from the central computing facility 214. Thus, the neurologist/physician may receive the kinetic state reporting by email or by being made available on a website or portal on the Internet or other communication network, in a format which can be rapidly interpreted by the neurologist to ensure efficient use of the neurologist's time. The neurologist 216 then interprets the report and updates the patient's medication or prescription or therapy so that it is optimised according to the objective clinical information contained in the report. Additionally or alternatively the neurologist may make recommendations, based on the report, for further evaluation using the inventive system or method e.g. to determine if the patient 212 is a candidate for a particular advanced therapy.

In this embodiment algorithms are applied to the obtained data by a central computing facility 214 in order to generate both a dyskinesia score and a bradykinesia. score for every 2 minute window of data, in the manner taught by WO 2009/149520.

The system 215 is shown in more detail in FIG. 2b. A nurse 210 or clinician may pre-screen candidates with reference to whether age is appropriate, cognitive impairment, and whether oral therapy (levodopa) is optimised. To undertake screening for advanced therapy according to embodiments of the invention, the nurse uses a tablet or similar device 220 having a processor executing a suitable application to configure the wrist worn motion monitor 121. Typically this involves the application executed by the device 220 receiving as inputs one or more of: a patient identifier, that patient's medication type and times, session coding details, and the like. The application creates a session key for encryption and decryption of data generated and transmitted through the system. The wrist worn motion monitor 121 is worn during typical daily activities of that patient 212, and during this period reminds the patient when to take medication, and in a preferred embodiment, receives patient input indicating when doses are taken. The motion monitor 115 may be removed when bathing (although a water resistant device is contemplated and within the scope of this disclosure) or sleeping or for recharging, then use may continue.

When the use session ends (typically after an extended period of 6 to 10 days or more), the motion monitor 115 is coupled with dock/charge station 222 and interfaced with a tablet or similar device 220 executing a suitable application for data retrieval from the docked motion monitor. The data is secured passed and (via wired or wireless means) to a clinic server or equivalently to the central computing facility 214 where patient specific data is retrieved by a processor of the central computing facility and analysed for calculation by the processor of a selection score according to embodiments of the present invention.

An example of the process 240 of data analysis undertaken by the inventive system 215 to produce the selection score (as may be included in a work product 225) is illustrated in FIG. 2c. As shown, an acceleration time series is used to derive DK and BK scores as described in WO 2009/149520.

In some embodiments, the central computing facility 214 further generates reports providing a more detailed interpretation of the retrieved and analysed data that is specific to the patient and provides clinical detail as to the factors underpinning the selection score as determined by the inventive system and method. The work product 225 generated by a processor of the central computing facility 214 or equivalent processer/server may include e.g. a PKG 226 and report 228 in PDF or other suitable file format readable by the clinician 216. An example of a work product 225 populated with meaningful clinical information is provided in FIG. 2d, Here, the work product template 225 includes a field for a PKG 226 of the patient (being a chart of data extracted from the motion monitor) and a report 228 including fields automatically populated by the system. Such fields may include but are not limited to: a patient identifier; referring clinician; duration of data collection; dates of data collection; dosage reminders provided to the subject; dosage acknowledgements (of medication dosages consumed) by the subject; therapies prescribed to the subject; summary of kinetic behaviour during data collection (including one or more of bradykinetic, dyskinetic and tremor motion); selection score as calculated by the system; summary of kinetic behaviour response to medication and in particular, evidence from the motion monitor data that the subject did or did not respond to medications such as levodopa based medications (or other medications); summary of clinical findings based on at least one of the motion data and measures of dispersion and selection score calculated by the processor. Advantageously, summary of kinetic behaviour response to medication including specific evidence from the motion monitor data that the subject did or did not respond to medications such as levodopa based medications (or other medications) is clinically valuable since levodopa responsiveness is a criteria for selection for advanced therapy.

In some embodiments, the system is configurable to collect data and/or populate fields in the work product template 225 representing metrics for clinical scales collected through the inventive system. These may include for example, cognitive measures or other measures including blood pressure, age, duration of disease, impulsivity or apathy. Such information may be collected via a patient portal provided by a device such as a tablet or mobile phone or other device operated by the patient or carer. Typically, the inventive system prompts the patient or carer to provide such information to the device. In some embodiments, the system is configurable to receive or prompt the patient or carer for further patient motion inputs pertaining to, for example, gait, as may be determined by the motion monitor or another body-worn device, or supplied by a nurse or clinician.

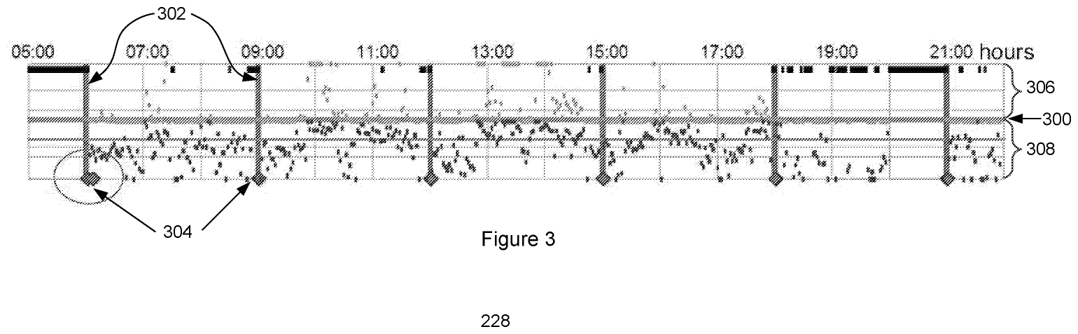

FIG. 3 gives an example of the output from one day of wrist-worn motion monitor recording from a patient who was prescribed 6 doses of levodopa per day. The upper set of data points 306 represent the dyskinesia scores (DK scores) produced from each 2 minute window of data, and the lower set of data points 308 represent the bradykinesia scores (BK scores) produced from each 2 minute window of data. DK scores are plotted only on or above the midline 300 of FIG. 3, while BK scores are plotted only on or below the midline 300 of FIG. 3. Greater severity of dyskinesia is represented by increasing distance of the DK scores 306 upwards from the midline, while greater severity of bradykinesia is represented by increasing distance of the BK scores 308 downwards from the midline. The horizontal lines indicate the respective median, 75th percentile and 90th percentile of controls, for both DK and BK scores, controls being subjects not having a neurodegenerative disorder. The six vertical lines, of which two are indicated at 302, indicate the times at which medications were prescribed, and the diamonds 304 represent when the taking of medication was acknowledged by the patient.

The present embodiment recognises that dispersion, or greater fluctuations, in the DK scores 306 and/or BK scores 308 over an extended period, is a useful predictor of whether motion symptoms have progressed to an advanced stage.

A study was conducted in which patients were monitored using the apparatus of FIG. 1 for 10 days, and all data was collected. The interquartile range for both the DK and BK scores is defined as the difference between (a) the value below which 75% of all data points fall and (b) the value below which 25% of all data points fall.

A measure of dispersion was computed in accordance with the fluctuation score taught by WO 2015/131244, the content of which is incorporated herein by reference. As noted in that disclosure a fluctuation score threshold of 7.7 is optimal under those study/population conditions. Using only the fluctuation score and a threshold of 7.7 gives rise to Sensitivity of 83%, selectivity of 47%, Fishers exact=p=0.078 and Kappa=0.304, while modifying the weights of the fluctuation score can provide an optimised fluctuation score having Sensitivity 84%: selectively 58% Fishers exact=p=0.038 and Kappa=0.364. However, the present invention recognises that the approach of the present invention can significantly improve the identification of DBS candidates.

The median BK scores correlate with contemporaneously obtained clinical ratings using the Unified Parkinson's rating Scale part III (UPDRS3), and the median DK scores correlate with clinically obtained ratings using the modified Abnormal Involuntary Movement Score (AIMS) rating.

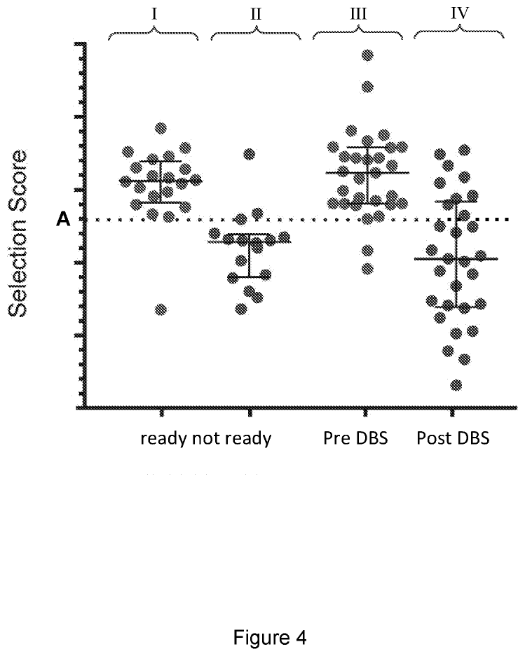

FIG. 4 illustrates classification efficacy of a selection score in accordance with one embodiment of the present invention. In this embodiment, the Selection Score was derived from the following inputs:

Selection Score=5.86*FDS+0.981+7.113K50+8,9* DK 50±6.913KS_{IQR}+8.8*Minutes_Under+4.04 *Minutes_Over+7.7*Reminder_Count>5+0.4*over count+−0.07*under count+0.4*tremor+−0.8*PTI+0.5*BK75.

Or, expressed in code form:

bin_score(n, bins)=findfirst(x−>n<x, [bins . . . ,Inf])−1dbss_a(x)=5.863*bin_score(x[TDS], [7.7, 9.4, 11.7])+7.136*bin_score(x[:BK_50], [22.0, 25.0, 31.0])+8.921*bin_score(x[:DK_50], [ 1.3, 3.0, 6.5])+6.957*bin_score(x[:BK_75]−x[:BK_25], [16.2, 19.1, 20.4])+8.805*bin_score(x[:Minutes_Under], [188, 288, 420])+4.041*bin_score(x[:Minutes_Over], [26, 95, 135])+7.737*bin_score(x[:Reminder_Count], [5])+−0.842*(x[:Minutes_Immobile]>z=54?−1:(x[:Minutes_Immobile]<=27?1:0))+0.433*x:[Over_Count]+−0,072*x:[Under_Count]+0,400*x[:Minutes_Tremor]

In FIG. 4 the first cluster of dots (I) represents patients who have a selection score above the threshold designated by line A and so have motion symptoms that are at an advanced stage. Here, line A is the best separation using ROC (other horizontal bars represent median, 25^{th} and 75^{th} percentiles for each group). The inventive system may therefore designate patients in the first cluster I as “ready” for an advanced therapy or as candidates for selection for advanced therapy. The second cluster of dots (II) represents patients who have a selection score near or below the threshold designated by line A and so have motion symptoms that are at an initial stage. The inventive system may therefore designate these patients as “not ready” for advanced therapy. This embodiment achieves high Sensitivity of 95% indicating that this selection score and system should identify most DBS candidates. This embodiment also provides high Specificity of 87.5%, showing that this method and system can tolerate false positives but increase specialty work load. This embodiment further provides a Fishers exact=p<0.0001 and Kappa=0.74.

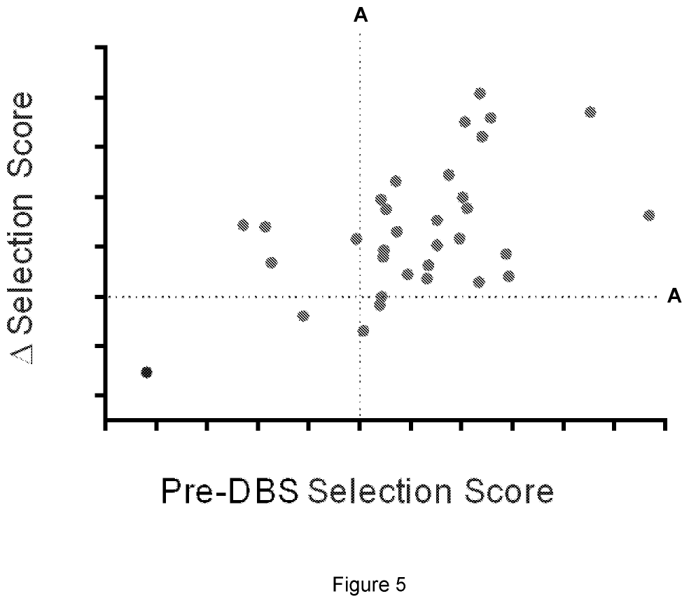

To further investigate efficacy of the Selection Score, the Selection Score was calculated on motion monitor data obtained from 33 people with Parkinson's (PwP) from four Australian centres, before (“pre DBS”) and six months after DBS (“post DBS”) as shown in clusters III and IV. Using the cut off threshold A, the Specificity in the cohort of cluster III was 90% and the Selectivity was 87.5%. It is relevant that one of the subjects with the lowest Selection Score was eventually diagnosed with multiple system atrophy (MSA) and should not in fact have been a DBS candidate. Furthermore the mean Selection Score fell by 25 points (p<0.0001, t-test) following DBS. As well there was broad tendency for those with the highest Selection Score to have the biggest improvement (FIG. 5).

Table 1 summarises the improved results delivered by the Selection Score as compared to two versions of the earlier Fluctuation Score (FS).

TABLE 1

| | Sensitivity | Selectivity | Fisher's | Kappa |
|---|---|---|---|---|
| Standard FS | 83% | 47% | p = 0.078 | 0.304 |
| Modified FS | 84% | 58% | p = 0.038 | 0.368 |
| Selection Score (FIG. 4) | 95% | 87.5% | <0.0001 | 0.74 |
| Re test of Selection | 90% | 87.5% | NA | NA |
| Score (cluster III) | | | | |

The next step was to use the study data set (FDS, BKS, DKS scores etc.) to train a decision system to predict which of the Australian PwP should have DBS. The system was again about 90% accurate (very similar to the Selection Score)

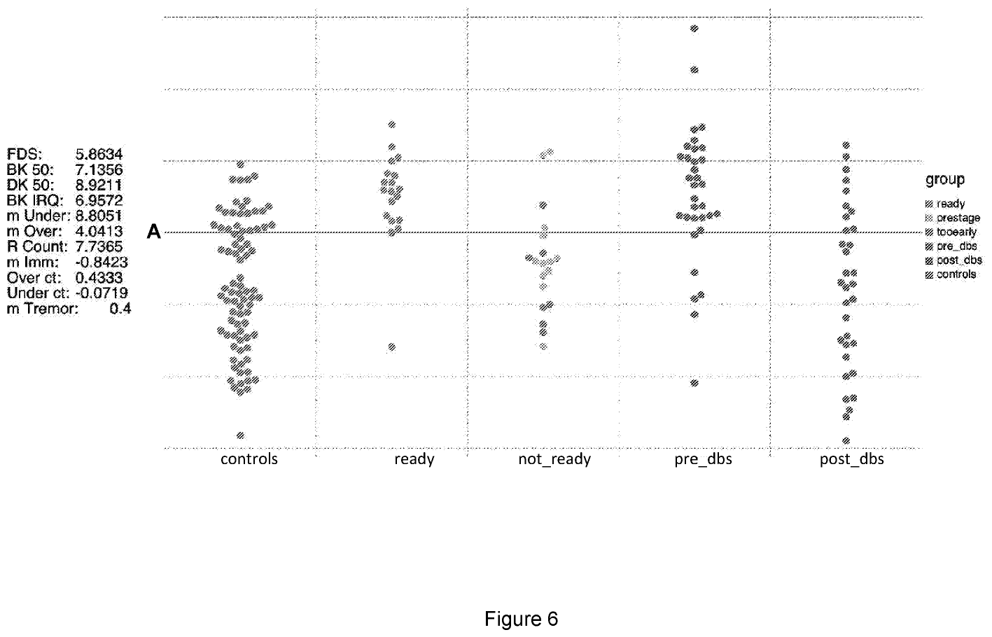

FIG. 6 further illustrates performance of the embodiment of FIGS. 4-5,

Thus it can be concluded that the Selection Score of this embodiment strongly predicts which patients require DBS,

Other embodiments of the Selection Score can be envisaged and are within the scope of the present invention.

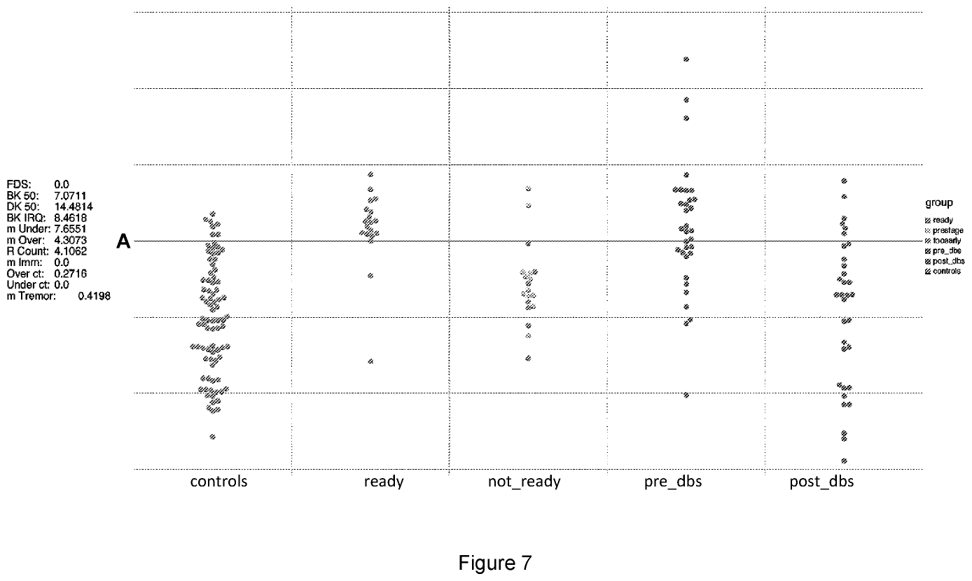

FIG. 7 illustrates an alternative embodiment of the invention, in which the Selection Score is determined from the following inputs:

Selection Score=6.8*FS+80*Reminder_Count>5+0.5*Minutes_Under+3.0*over count

Or, expressed in code form:

dbss_b(x)=7.071*bin_score(x[:BK_50], [22.0, 25.0, 31.0])+14.481*bin_score(x[:DK_50], [1.3, 3.0, 6.5])+8.462*bin_score(x[:BK_75]−x[:BK_25], [16.2, 19.1, 20.4])+7.655*bin_score(x[:Minutes_Under], [188, 288, 420])+4.307*bin_score(x[:Minutes_Over], [ 26, 95, 135])+4.106*bin_score(x[:Remincler_count], [5 ])+0.272*x[:Over Count]0.420*x[:Minutes_Tremor]

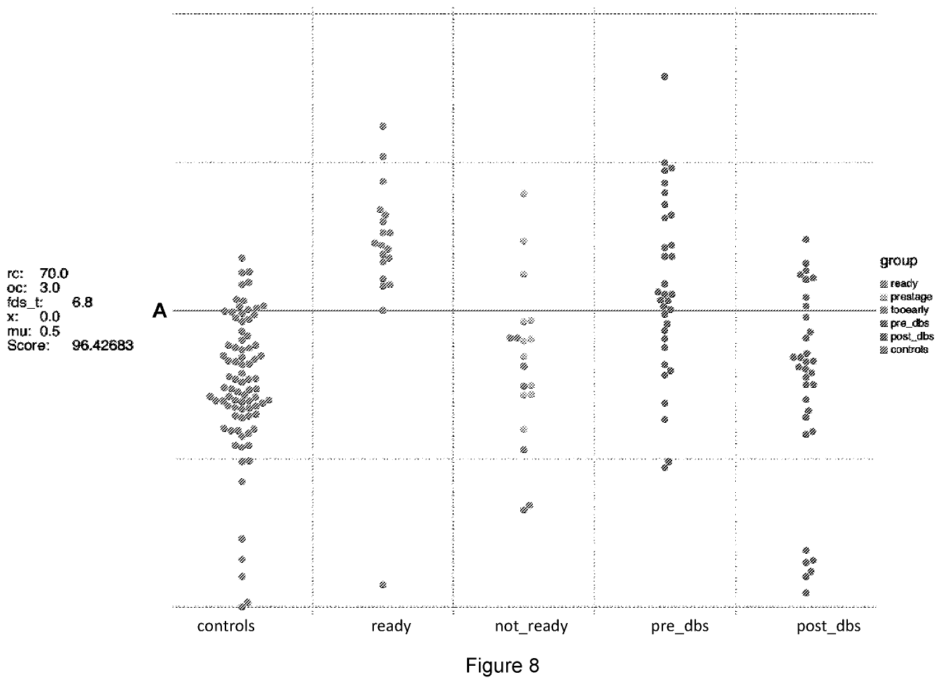

FIG. 8 illustrates another alternative embodiment of the invention, in which the Selection Score is determined from the following inputs:

Selection Score=0.7.5*FDS+0.981*DK_50+0.202*BKSIQR+2.885*Minutes_Under+1.743 Minutes_Over+1.154*Reminder_Count>5]

Or, expressed in code form:

dbss_s(x)=(x[:FDS]>6.8?100:0)+(x[:Reminder_Count]>5.0?70:0)+x[:Minutes_Under]*0.5+x[:Over_Count]*3.0

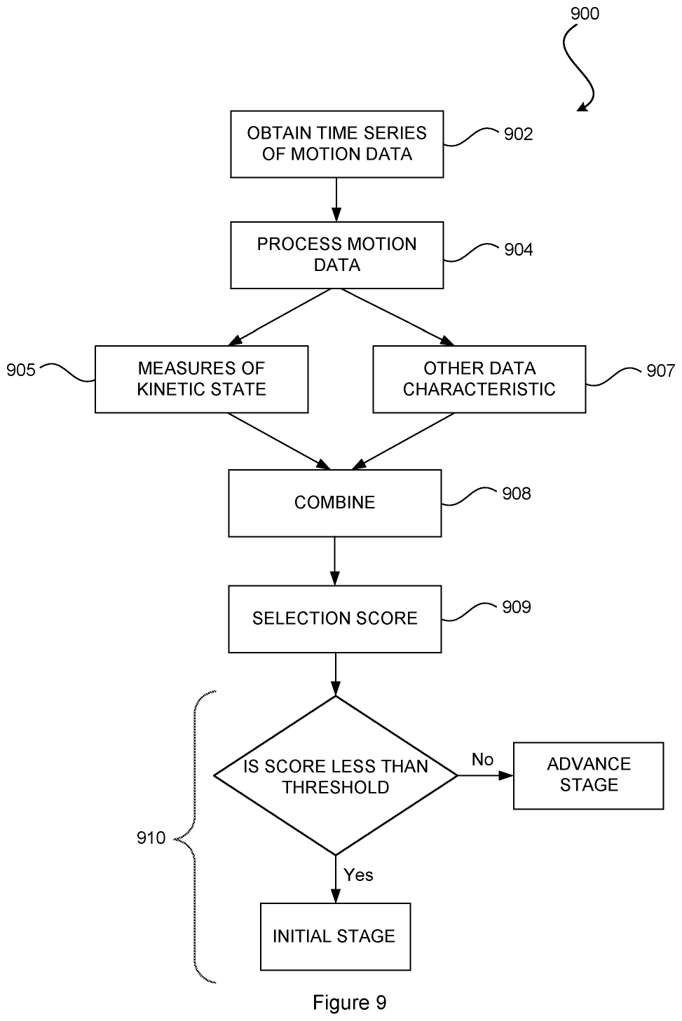

FIG. 9 illustrates schematically steps in a method 900 for method of determining a state of progression in an subject of a disease or treatment having motion symptoms, In a step 902 a processor obtains a time series of motion data from a motion detector worn on an extremity of the subject, over an extended period during usual activities of the subject. In a step 904 the processor processes the motion data to produce a plurality of measures of kinetic state (905) of the subject at a respective plurality of times throughout the extended period, each measure of kinetic state comprising at least one of: a measure for bradykinesia, and a measure for dyskinesia. The processor also determines at least one other data characteristic (907) determined from the motion data. In a step 908 the processor determines a measure of dispersion of the measures of kinetic state and in a step 908 combines the measure of dispersion with the one or more other data characteristic to produce a selection score (909). In some embodiments, the processor generates an output indicative of the selection score (909). In some embodiments, in a step 910 the processor generates an output indicating that motion symptoms are at an initial stage if the selection score is less than a threshold, and generating an output indicating that motion symptoms are at an advanced stage if the selection score is greater than the threshold.

In summary, it is proposed that a Selection Score derived from dispersion, and also from other data characteristics of a movement symptoms data series, has the potential to be used as an improved tool for choosing and optimising therapies for patients with motion disorders including PD. This invention recognises that in the conventional approach to advanced therapy, an experienced clinician assesses several symptoms to recognise a suitable DBS candidate based on their experience and knowledge. These symptoms might include overall level of bradyki nesi a and dyskinesia, the amount of time spent in dyskinesia and bradykinesia, the number of medication doses, cognition and the variability of kinetic state.

The present invention provides for collection and machine analysis of one or more of several other data characteristics, such as number of minutes during an observation period when the patient was not dyskinetic or when dyskinesia was below a threshold and when the subject was not bradykinetic or when bradykinesia. was below a threshold, as well as other patient data such as years with PD or other motion disorder, cognitive state, blood pressure, impulsivity, apathy and the like to produce a robust selection score indicative of the stage of progression of the disease or therapy. The selection score of the present invention is particularly effective as it objective and accommodates more than one kinetic symptom, and gathers much more detailed and accurate kinetic state data than patient diaries.

Some portions of this detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

As such, it will be understood that such acts and operations, which are at times referred to as being computer-executed, include the manipulation by the processing unit of the computer of electrical signals representing data in a structured form. This manipulation transforms the data or maintains it at locations in the memory system of the computer, which reconfigures or otherwise alters the operation of the computer in a manner well understood by those skilled in the art. The data structures where data is maintained are physical locations of the memory that have particular properties defined by the format of the data. However, while the invention is described in the foregoing context, it is not meant to be limiting as those of skill in the art will appreciate that various of the acts and operations described may also be implemented in hardware.

It should be borne in mind, however, that all of these and similar terms are to be associated with the appropriate physical quantities and are merely convenient labels applied to these quantities. Unless specifically stated otherwise as apparent from the description, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present invention also relates to apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, and each coupled to a computer system bus.

The algorithms and displays presented herein are not inherently related to any particular computer or other apparatus. Various general purpose systems may be used with programs in accordance with the teachings herein, or it may prove convenient to construct more specialized apparatus to perform the required method steps. The required structure for a variety of these systems will appear from the description. In addition, the present invention is not described with reference to any particular programming language. It will be appreciated that a variety of programming languages may be used to implement the teachings of the invention as described herein.

A machine-readable medium includes any mechanism for storing or transmitting information in a form readable by a machine (e.g., a computer). For example, a machine-readable medium includes read only memory (“ROM”); random access memory (“RAM”); magnetic disk storage media; optical storage media; flash memory devices; electrical, optical, acoustical or other form of propagated signals (e.g., carrier waves, infrared signals, digital signals, etc.); etc.

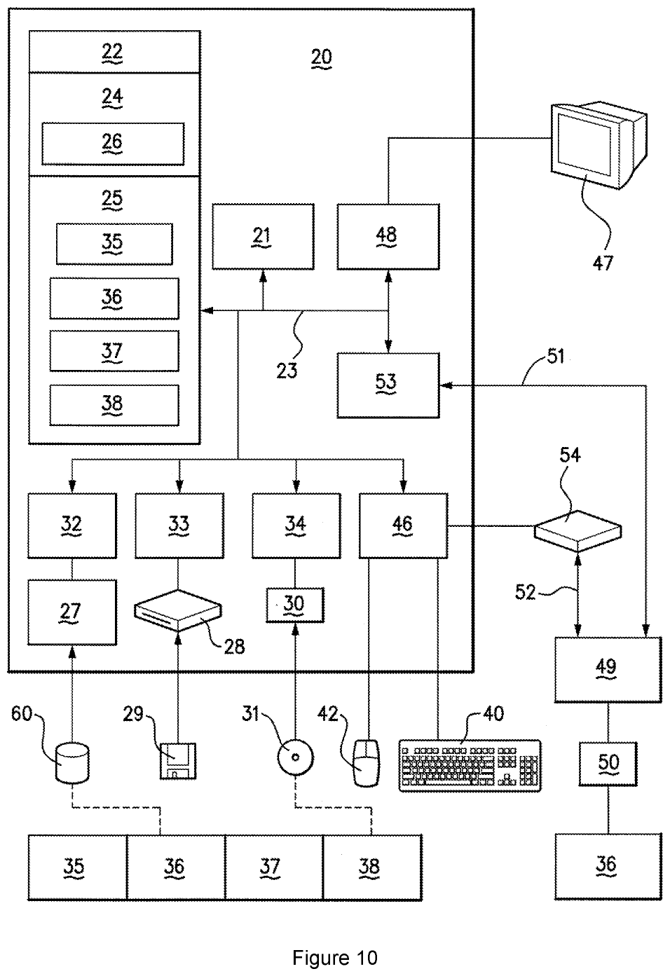

Turning to FIG. 10, the invention is illustrated as being implemented in a suitable computing environment. Although not required, the invention will be described in the general context of computer-executable instructions, such as program modules, being executed by a personal computer. Generally, program modules include routines, programs, objects, components, data structures, etc. that perform particular tasks or implement particular abstract data types. Moreover, those skilled in the art will appreciate that the invention may be practiced with other computer system configurations, including hand-held devices, multi-processor systems, microprocessor-based or programmable consumer electronics, network PCs, minicomputers, mainframe computers, and the like. The invention may he practiced in distributed computing environments where tasks are performed by remote processing devices that are linked through a communications network. In a distributed computing environment, program modules may be located in both local and remote memory storage devices.

In FIG. 10 a general purpose computing device is shown in the form of a conventional personal computer 20, including a processing unit 21, a system memory 22, and a system bus 23 that couples various system components including the system memory to the processing unit 21. The system bus 23 may be any of several types of bus structures including a memory bus or memory controller, a peripheral bus, and a local bus using any of a variety of bus architectures. The system memory includes read only memory (ROM) 24 and random access memory (RAM) 25. A basic input/output system (BIOS) 26, containing the basic routines that help to transfer information between elements within the personal computer 20, such as during start-up, is stored in ROM 24. The personal computer 20 further includes a hard disk drive 27 for reading from and writing to a hard disk 60, a magnetic disk drive 28 for reading from or writing to a removable magnetic disk 29, and an optical disk drive 30 for reading from or writing to a removable optical disk 31 such as a CD ROM or other optical media.

The hard disk drive 27, magnetic disk drive 28, and optical disk drive 30 are connected to the system bus 23 by a hard disk drive interface 32, a magnetic disk drive interface 33, and an optical disk drive interface 34, respectively. The drives and their associated computer-readable media provide nonvolatile storage of computer readable instructions, data structures, program modules and other data for the personal computer 20. Although the exemplary environment shown employs a hard disk 60, a removable magnetic disk 29, and a removable optical disk 31, it will be appreciated by those skilled in the art that other types of computer readable media which can store data that is accessible by a computer, such as solid state drives (SSDs) magnetic cassettes, flash memory cards, digital video disks, Bernoulli cartridges, random access memories, read only memories, storage area networks, and the like may also be used in the exemplary operating environment.

A number of program modules may be stored on the hard disk 60, magnetic disk 29, optical disk 31, ROM 24 or RAM 25, including an operating system 35, one or more applications programs 36, other program modules 37, and program data 38. A user may enter commands and information into the personal computer 20 through input devices such as a keyboard 40 and a pointing device 42. Other input devices (not shown) may include a microphone, joystick, game pad, satellite dish, scanner, or the like. These and other input devices are often connected to the processing unit 21 through a serial port interface 46 that is coupled to the system bus, but may be connected by other interfaces, such as a parallel port, game port or a universal serial bus (USB) or a network interface card. A monitor 47 or other type of display device is also connected to the system bus 23 via an interface, such as a video adapter 48. In addition to the monitor, personal computers typically include other peripheral output devices, not shown, such as speakers and printers.

The personal computer 20 may operate in a networked environment using logical connections to one or more remote computers, such as a remote computer 49. The remote computer 49 may be another personal computer, a server, a router, a network PC, a peer device or other common network node, and typically includes many or all of the elements described above relative to the personal computer 20, although only a memory storage device 50 has been illustrated. The logical connections depicted include a local area network (LAN) 51 and a wide area network (WAN) 52. Such networking environments are commonplace in offices, enterprise-wide computer networks, intranets and, inter alia, the Internet.

When used in a LAN networking environment, the personal computer 20 is connected to local network 51 through network interface or adapter 53. When used in a WAN networking environment, the personal computer 20 typically includes modem 54 or other means for establishing communications over WAN 52. The modem 54, which may be internal or external, is connected to system bus 23 via the serial port interface 46. In a networked environment, program modules depicted relative to the personal computer 20, or portions thereof, may be stored in the remote memory storage device. It will be appreciated that the network connections shown are exemplary and other means of establishing a communications link between the computers may be used.

Also described herein is a method of determining a state of progression in a subject of a disease or treatment having motion symptoms, the method comprising:

obtaining a time series of motion data from a motion detector worn on an extremity of the subject, over an extended period during usual activities of the subject;

processing the motion data to produce a plurality of measures of kinetic state of the subject at a respective plurality of times throughout the extended period, each measure of kinetic state comprising at least one of: a measure for bradykinesia, and a measure for dyskinesia;

determining a measure of dispersion of the measures of kinetic state;

combining the measure of dispersion with at least one other data characteristic determined from the motion data, to produce a selection score; and

generating an output indicating that motion symptoms are at an initial stage if the selection score is less than a threshold, and generating an output indicating that motion symptoms are at an advanced stage if the selection score is greater than the threshold.

Also described herein is a non-transitory computer readable medium for determining a state of progression in a subject of a disease or treatment having motion symptoms, comprising instructions which, when executed by one or more processors, causes performance of the following:

obtaining a time series of motion data from a motion detector worn on an extremity of the subject, over an extended period during everyday activities of the subject;

processing the motion data to produce a plurality of measures of kinetic state of the subject at a respective plurality of times throughout the extended period, each measure of kinetic state comprising at least one of: a measure for bradykinesia, and a measure for dyskinesia;

determining a measure of dispersion of the measures of kinetic state;

combining the measure of dispersion with at least one other data characteristic determined from the motion data, to produce a selection score; and

generating an output indicating that motion symptoms are at an initial stage if the selection score is less than a threshold, and generating an output indicating that motion symptoms are at an advanced stage if the selection score is greater than the threshold.

Also described herein is a system for determining a state of progression in a subject of a disease or treatment having motion symptoms, the system comprising:

a motion detector configured to be worn on an extremity of the subject and to output a time series of motion data over an extended period; and

a processor configured to receive the motion data and to process the motion data to produce a plurality of measures of kinetic state of the subject at a respective plurality of times throughout the extended period, each measure of kinetic state comprising at least one of: a measure for bradykinesia, and a measure for dyskinesia; the processor further configured to determine a measure of dispersion of the measures of kinetic state; the processor further configured to combine the measure of dispersion with at least one other data characteristic determined from the motion data, to produce a selection score; and the processor further configured to generate an output indicating that motion symptoms are at an initial stage if the selection score is less than a threshold, and to generate an output indicating that motion symptoms are at an advanced stage if the selection score is greater than the threshold.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of determining a state of progression in a subject of a disease or treatment having motion symptoms, the method comprising;
obtaining a time series of motion data from a motion detector worn on an extremity of the subject, over an extended period during usual activities of the subject;
processing the motion data to produce a plurality of measures of kinetic state of the subject at a respective plurality of times throughout the extended period, each measure of kinetic state comprising at least one of: a measure for bradykinesia, and a measure for dyskinesia;
determining a measure of dispersion of the measures of kinetic state;
combining the measure of dispersion with at least one other data characteristic determined from the motion data, to produce a selection score; and
generating an output indicating the selection score.

2. The method according to claim 1, including the step of generating an output indicating that motion symptoms are at an initial stage if the selection score is less than a threshold, and generating an output indicating that motion symptoms are at an advanced stage if the selection score is greater than the threshold.

3. The method according to claim 2, wherein the measure of dispersion is one of:
a measure of the numerical distance between a high and low percentile of the measures of kinetic state;
a measure of the interquartile range of the measures of kinetic state;
a measure of the variance of the measures of kinetic state; and
at least one of a measure of the standard deviation of the measures of kinetic state, an indicator of the variability of the measures of kinetic state, an indicator of the scatter of the measures of kinetic state, and an indicator of the spread of the measures of kinetic state.

4. (canceled)

5. (canceled)

6. (canceled)

7. The method according to claim 1, wherein the at least one other data characteristic comprises one or more of:
a probabilistic measure of bradykinesia;
a probabilistic measure of dyskinesia;
a median or mean DK score in a period where the subject is ‘off’;
minutes OFF, being the number of minutes during an observation period when the subject was not dyskinctic or when dyskinesia was below a threshold;
minutes in dyskinesia, being the number of minutes during an observation period when the subject was dvskinetic or when dyskinesia was above a threshold;
minutes in bradvkinesia, being the number of minutes during an observation period when the subject was bradvkinetic or when bradvkinesia was above a threshold;
the proportion of time immobile (PTI) or amount of time immobile (ATI);
a measure of tremor derived from the motion data;
BKS_{IOR}, being die interquartile range of BK scores:
a measure of the number of windows of time throughout the observation period in which at least 5 out of 7 DK scores exceed the 75th percentile;
a measure of the number of windows of time throughout the observation period in which at least 5 out of 7 BK scores exceed the 75th percentile.

8. The method according to claim 7 wherein the probabilistic measure of bradykinesia comprises one or more of:
a mean or median value of a time series of individual measures of bradykinesia obtained throughout an observation period; and
a percentile value of a time series of individual measures of bradykinesia; and
a 75^{th} percentile value of a time series of individual measures of bradykinesia.

9. (canceled)

10. (canceled)

11. (canceled)

12. The method according to claim 7 wherein the probabilistic measure of dyskinesia comprises one or more of:
a mean or median value of a time series of individual measures of dyskinesia obtained throughout an observation period;
a percentile value of a time series of individual measures of dyskinesia; and
a 75^{th} percentile value of a time series of individual measures of dyskinesia.

13. (canceled)

14. (canceled)

15. (canceled)

16. (canceled)

17. (canceled)

18. (canceled)

19. The method according to claim 1 further including combining the measure of dispersion with at least one other data characteristic, to produce the selection score; and wherein the at least one other data characteristic comprises a dosage measure.

20. The method according to claim 19 wherein the dosage measure comprises a number of medication reminders prescribed for that subject during a period of interest.

21. (canceled)

22. (canceled)

23. (canceled)

24. (canceled)

25. (canceled)

26. The method according to claim 1 wherein the motion data is obtained only during waking hours.

27. The method according to claim 1 wherein each measure of kinetic state comprises both a measure for bradykinesia and a measure for dyskinesia.

28. The method according to claim 27 wherein each measure of dispersion is produced as a weighted sum of a measure of the dispersion of the measures for bradykinesia and a measure of the dispersion of the measures for dyskinesia.

29. The method according to claim 1 wherein the measure of dispersion is produced by summing each measure of bradykinesia with a contemporaneous measure of dyskinesia to produce a combined measure of kinetic state, and determining the measure of dispersion from the dispersion of the combined measures of kinetic state.

30. The method according to claim 1 further comprising recording a value of the selection score as determined on different occasions in order to monitor progression of the selection score, for example over the course of hours, days, weeks, months or years: and optionally, monitoring a rate of change in the selection score over time to project or predict disease progression towards a threshold at which advanced therapies may become appropriate.

31. (canceled)

32. The method according to claim wherein the monitoring of the selection score during progression of a disease is used as a basis to indicate which therapy, of a plurality of available progressions in therapy, is suitable for that particular subject.

33. The method according to claim 1 further comprising one or more of:
aggregating selection scores obtained for a plurality of subjects in order to assess a state or progression of disease or treatment of the group;
combining the measure of dispersion with at least one other data characteristic not derived from motion data, to produce the selection score, wherein the at least one other data characteristic is selected from the group including:
number of medication reminders provided to a subject:
dosage acknowledgements by the subject;
years with motion disease;
subject's cognitive state;
subject's age:
blood pressure;
impulsivity; and
apathy; and
automatically generating a subject specific report based on a report module containing instructions executable by a processor receiving at least the motion data, wherein the report module populates fields of a report template with the selection score and clinical observations derived front the motion data.

34. (canceled)

35. (canceled)

36. The method according to claim wherein the report template includes fields selected from the group including: a subject identifier; referring clinician;
duration of data collection; dates of data collection; dosage acknowledgements by the subject;
therapies prescribed to the subject; dosage reminders provided to the subject; summary of kinetic behaviour during data collection (including one or more of bradykinetic, dyskinetic and tremor motion); summary of kinetic behaviour response to medication; and summary of clinical findings based on at least one of the motion data and measures of dispersion and selection score calculated by the processor.

37. A non-transitory computer readable medium for determining a state of progression in a subject of a disease or treatment having motion symptoms, comprising instructions which, when executed by one or more processors, causes performance of the following:
obtaining a time series of motion data from a motion detector worn on an extremity of the subject, over an extended period during everyday activities of the subject;
processing the motion data to produce a plurality of measures of kinetic state of the subject at a respective plurality of times throughout the extended period, each measure of kinetic state comprising at least one of: a measure for bradykinesia, and a measure for dyskinesia;
determining a measure of dispersion of the measures of kinetic state;
combining the measure of dispersion with at least one other data characteristic determined from the motion data, to produce a selection score; and
generating an output indicating the selection score.

38. A non-transitory computer readable medium according to claim 37 comprising instructions which cause performance of the step of generating an output indicating that motion symptoms are at an initial stage if the selection score is less than a threshold, and generating an output indicating that motion symptoms are at an advanced stage if the selection score is greater than the threshold.

39. A system for determining a state of progression in a subject of a disease or treatment having motion symptoms, the system comprising:
a motion detector configured to be worn on an extremity of the subject and to output a time series of motion data over an extended period; and
a processor configured to receive the motion data and to process the motion data to produce a plurality of measures of kinetic state of the subject at a respective plurality of times throughout the extended period, each measure of kinetic state comprising at least one of: a measure for bradykinesia, and a measure for dyskinesia; the processor further configured to determine a measure of dispersion of the measures of kinetic state; the processor further configured to combine the measure of dispersion with at least one other data characteristic determined from the motion data, to produce a selection score; and the processor further configured to generate an output indicating the selection score.

40. The system according to claim 39 wherein the processor is configured to generate an output indicating that motion symptoms are at an initial stage if the selection score is less than a threshold, and to generate an output indicating that motion symptoms are at an advanced stage if the selection score is greater than the threshold.

41. (canceled)

42. A method for automated screening of a subject to determine clinical readiness to receive advanced therapy for a disease having motion symptoms, the method comprising:
obtaining at a processor a time series of motion data from a motion detector worn on an extremity of the subject, over an extended period during usual activities of the subject;
the processor calculating from the motion data a plurality of measures of kinetic state of the subject at a respective plurality of times throughout the extended period, each measure of kinetic state comprising at least one of: a measure for bradykinesia, and a measure for dyskinesia;
the processor determining a measure of dispersion of the measures of kinetic state; and
the processor combining the measure of dispersion with at least one other data characteristic determined from the motion data, to produce a selection score; and
the processor generating an output indicating one or more of clinical readiness for advanced therapy when the selection score is greater than a threshold; and clinical unreadiness for advanced therapy when the selection score is less than the threshold.

43. The method according to claim 42, wherein the threshold is selected from the group including:
(i) a median level of the selection score for subjects having received advanced therapy;
(ii) the 75th percentile level of the selection score for subjects having received advanced therapy; and
(iii) a scalar, logarithmic or exponential variant derived from such values in (i) or (ii).

44. The method according to claim 42, further including the step of automatically determining a subject's readiness to receive an advanced therapy selected from the group including: deep brain stimulation (DBS), apomorphine and levodopa-carbidopa (duodopa), wherein the subject's readiness to receive the selected advanced therapy is automatically determined by (he processor when (he selection score is greater than a threshold determined by a median level of the selection score for subjects having received the selected advanced therapy, or the 75th percentile level of the selection score for subjects having received the selected advanced therapy or an aggregate of these, or a scalar, logarithmic or exponential variant derived from such values.

45. (canceled)

46. The method according to claim 42 , further comprising automatically generating a subject specific report based on a report module containing instructions that are executable by the processor, wherein the report module populates fields of a report template with the selection score and clinical observations derived from the motion data.

47. (canceled)
